(19) United States
(12) Patent Application Publication
(10) Pub. No.: US 2026/0263247 A1
(43) Pub. Date: Sep. 10, 2026

(54) HANDLE FOR STENT DELIVERY SYSTEM

(71) Applicant: BOSTON SCIENTIFIC SCIMED, INC., MAPLE GROVE, MN (US)

(72) Inventors: Gary Gilmartin, Foxford (IE); Fionn Stapleton, Oranmoe (IE); Daniel Tuck, Oughterard (IE); David Giles, Galway (IE); Keith O'Reilly, Kilcolgan (IE); Shane O'Keeffe, Tuam (IE)

(73) Assignee: BOSTON SCIENTIFIC SCIMED, INC., MAPLE GROVE, MN (US)

(21) Appl. No.: 19/552,325

(22) Filed: Feb. 27, 2026

Related U.S. Application Data
(60) Provisional application No. 63/767,699, filed on Mar. 6, 2025.

(51) Int. Cl. A61F 2/95; A61F 2/966; A61F 2/97

(52) U.S. Cl. CPC A61F 2/9517; A61F 2/966; A61F 2/97

(57) ABSTRACT

A stent delivery system includes a handle having a lumen and a stop disposed within the lumen. A stent delivery catheter includes an inner shaft positioned within the handle lumen with its proximal end abutting the stop, and an outer tubular shaft slidably disposed over the inner shaft. The outer tubular shaft is configured to be retracted proximally past the stop. The system enables controlled deployment of self-expanding stents through relative movement between the inner and outer shafts.

### CROSS REFERENCE TO RELATED APPLICATIONS

The present application claims the benefit of US Provisional Patent Application Serial No. 63/767,699, filed Mar. 6, 2025, the disclosure of which is incorporated herein by reference.

### TECHNICAL FIELD

The present disclosure generally relates to deployment of self-expanding stents in co-axial stent delivery systems. More specifically, the present disclosure is directed to stent delivery systems having a handle stop attachment configured to improve deployment accuracy.

### BACKGROUND

In a co-axial stent deployment system, a catheter shaft, which may be inserted through the working channel of an endoscope in some instances, may include an outer shaft slidably disposed over an inner shaft. To deploy a self-expanding stent therefrom, the outer shaft is withdrawn proximally relative to the inner shaft to expose the stent from the distal end of the outer shaft, thereby allowing the stent to radially expand from a radially constrained configuration within the outer shaft.

However, movements extraneous to the proximal withdrawal of the outer shaft may cause the stent to be released prematurely and/or off-target. Thus, to reduce inaccuracies in stent deployment, a system that would fix the position of the inner shaft as to enable controlled retraction of the outer shaft is desired.

### BRIEF SUMMARY

This disclosure provides design, material, manufacturing method, and use alternatives for medical devices, including stent delivery and/or deployment devices.

A first example is a stent delivery system. The system includes a handle having a proximal end and a distal end. The handle includes a handle casing having an interior surface defining a lumen along a longitudinal axis of the handle casing, a stop disposed within the lumen of the handle casing, and at least one strut supporting the stop within the lumen of the handle casing such that an outer circumferential surface of the stop is spaced radially inward of and spaced away from the interior surface of the handle casing. The system also includes a stent delivery catheter. The stent delivery catheter includes an inner shaft having a proximal end region configured to be positioned within the lumen of the handle casing with a proximal end of the inner shaft abutting a distal facing surface of the stop, and an outer tubular shaft slidably disposed over the inner shaft, the outer tubular shaft including a proximal end region configured to be positioned within the lumen of the handle casing. Proximal retraction of the outer tubular shaft causes a proximal end of the outer tubular shaft to be retracted proximal of the stop.

Alternatively or additionally to any of the examples herein, in another example, the strut comprises a blade configured to cut the outer tubular shaft when the outer tubular shaft is retracted proximally relative to the inner shaft.

Alternatively or additionally to any of the examples herein, in another example, the outer tubular shaft includes at least one longitudinal slit corresponding to a position of the strut.

Alternatively or additionally to any of the examples herein, in another example, the system, such as the handle thereof, includes a retraction member coupled to the outer tubular shaft.

Alternatively or additionally to any of the examples herein, in another example, the retraction member comprises a sliding knob coupled to the outer tubular shaft by a clamp.

Alternatively or additionally to any of the examples herein, in another example, the stop comprises an inner ring including a central opening configured to receive a guidewire therethrough.

Alternatively or additionally to any of the examples herein, in another example, the handle casing comprises a distal end configured to be secured to a working channel of an endoscope.

Alternatively or additionally to any of the examples herein, in another example, the handle casing is configured for lateral placement of the stent delivery catheter therein.

Alternatively or additionally to any of the examples herein, in another example, the stop has a C-shaped cross-section and a lateral opening.

Alternatively or additionally to any of the examples herein, in another example, the catheter shaft is configured to be cut to adjust its length.

Alternatively or additionally to any of the examples herein, in another example, the at least one strut comprises two diametrically opposed struts.

Alternatively or additionally to any of the examples herein, in another example, the stop includes a support ring secured to the handle casing.

Alternatively or additionally to any of the examples herein, in another example, the at least one strut supports an inner ring of the stop coaxially within the support ring.

Alternatively or additionally to any of the examples herein, in another example, an outer circumferential surface of the inner ring is spaced apart from an inner circumferential surface of the support ring, defining an annular gap therebetween.

Another example is a stent delivery system. The system includes a handle having a proximal end and a distal end. The handle includes a handle casing having an interior surface defining a lumen along a longitudinal axis of the handle casing, an inner ring disposed within the lumen of the handle casing, the inner ring including a central opening configured to receive a guidewire therethrough, and at least one strut supporting the inner ring within the lumen of the handle casing such that an outer diameter of the inner ring is spaced radially inward of the inner surface of the handle casing. The system also includes a stent delivery catheter. The stent delivery catheter includes an inner shaft and an outer tubular shaft slidably disposed over the inner shaft. A proximal end of the inner shaft is configured to abut the inner ring within the lumen of the handle casing. The outer tubular shaft is configured surround the inner ring when the outer tubular shaft is retracted proximally relative to the inner shaft.

Alternatively or additionally to any of the examples herein, in another example, the strut comprises a blade configured to cut the outer tubular shaft when the outer tubular shaft is retracted proximally relative to the inner shaft.

Alternatively or additionally to any of the examples herein, in another example, the outer tubular shaft includes at least one pre-formed longitudinal slit corresponding to a position of the strut, configured to allow the outer tubular shaft to pass over the inner ring during proximal retraction of the outer tubular shaft relative to the inner shaft.

Alternatively or additionally to any of the examples herein, in another example, the system, such as the handle thereof, includes a support ring secured to the handle casing, wherein the at least one strut supports the inner ring coaxially within the support ring.

Alternatively or additionally to any of the examples herein, in another example, a proximal end of the inner shaft is configured to abut the inner ring within the lumen of the handle casing.

Another example is a method of deploying a stent. The method includes advancing a catheter shaft to a treatment site through a working channel of an endoscope, wherein a proximal end region of the catheter shaft extends proximally from the working channel of the endoscope. The catheter shaft includes an inner shaft having a distal end, a proximal end, and a lumen configured to receive a guidewire. The catheter shaft further includes an outer tubular shaft having a distal end and a proximal end. The outer tubular shaft is slidably disposed over the inner shaft. The outer tubular shaft is configured to hold a stent in a constrained position on the distal end of the inner shaft. The method further includes positioning the proximal end region of the catheter shaft into a lumen of a handle casing of a handle such that the proximal end of the inner shaft abuts a distally facing stop within the lumen of the handle casing. The method further includes securing a distal end of the handle casing to the working channel of the endoscope, and retracting the outer tubular shaft proximally relative to the inner shaft to deploy the stent.

Alternatively or additionally to any of the examples herein, in another example, the method includes intraoperatively shortening the length of the catheter shaft, such as cutting off a proximalmost length of the catheter shaft (e.g., cutting off a proximalmost length of the inner shaft and/or the outer tubular shaft) prior to position the proximal end region of the catheter shaft into the lumen of the handle casing of the handle.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE SEVERAL VIEWS OF THE DRAWINGS

FIG. 1 is a perspective cross-sectional view of an exemplary stent delivery system in accordance with an embodiment disclosed herein.

FIG. 2 is an enlarged view of a portion of the exemplary stent delivery system of FIG. 1.

FIG. 3A is a partial cross-sectional side view of an exemplary stent delivery system in a pre-deployment position, in accordance with an embodiment of the subject matter disclosed herein.

FIG. 3B is a partial cross-sectional side view of an alternative configuration of exemplary stent delivery system in a pre-deployment position, in accordance with an embodiment of the subject matter disclosed herein.

FIG. 3C is a partial cross-sectional side view of an exemplary stent delivery system in a post-deployment position, in accordance with an embodiment of the subject matter disclosed herein.

FIG. 4 is a perspective view of an exemplary stent delivery system with a partial cutaway in accordance with an embodiment of the subject matter disclosed herein.

FIG. 5A is a perspective view of an exemplary stent delivery system with a partial cutaway in accordance with an embodiment of the subject matter disclosed herein.

FIG. 5B is a perspective view of an alternative configuration of an exemplary stent delivery system with a partial cutaway in accordance with an embodiment of the subject matter disclosed herein.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

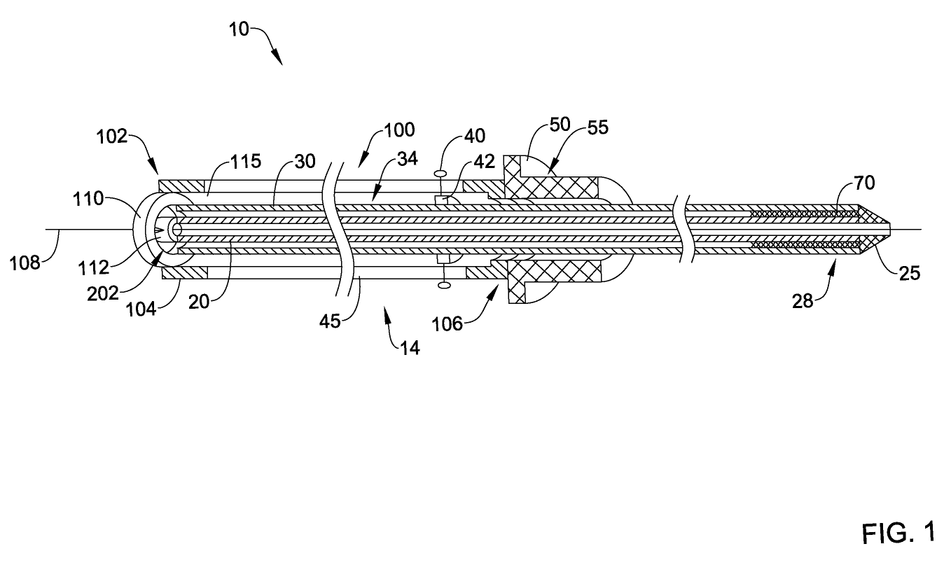

FIG. 1 shows a perspective view of a stent delivery system 10. Stent delivery system 10 includes a handle 100 and a stent delivery catheter 12 (herein referred to as “catheter”). The catheter 12 may include an inner shaft 20 and an outer tubular shaft 30 surrounding the inner shaft 20. The outer tubular shaft 30 is slidably disposed over the inner shaft 20 with the inner shaft 20 extending through a lumen of the outer tubular shaft 30. The catheter 12 may include a distal tip 25 located at the distal end of the inner shaft 20. A self-expanding stent 70 is compressably constrained by the outer tubular shaft 30 at the distal end region 28 of the inner shaft 20. For example, the self-expanding stent 70 may be radially constrained around the distal end region 28 of the inner shaft 20 with the self-expanding stent 70 positioned radially between an inner surface of the outer tubular shaft 30 and the outer surface of the inner shaft 20. The self-expanding stent 70 may be deployed from the catheter 12 through proximal retraction of the outer tubular shaft 30 relative to the inner shaft 20 and the self-expanding stent 70, whereby the self-expanding stent 70 is able to revert to a radially enlarged deployment configuration once unconstrained by the outer tubular shaft 30. In some instances, the inner shaft 20 may include a lumen configured to receive a guidewire 108. The guidewire 108 may be used to provide a path for the advancement of the stent delivery system 10 through a body lumen, position the stent 70 at the target site, and otherwise help in accurate delivery and deployment.

Stent delivery system 10 may include a handle 100, with a distal end106 and proximal end 102, to aid in deployment of the stent 70. The handle 100 includes a handle casing 104, which has an interior surface that defines a lumen 115 extending along the longitudinal axis of the handle casing 104. The handle 100 may be configured to surround a proximal end region of the catheter 12, including a proximal end region of the inner shaft 20 and a proximal end region of the outer tubular shaft 30. Accordingly, the proximal end region of the catheter 12, including the proximal end region of the inner shaft 20 and the proximal end region of the outer tubular shaft 30 may extend into the lumen 115 of the handle casing 104 from the distal end 106 of the handle 100.

To deliver and deploy the self-expanding stent 70, the catheter 12 may be advanced into a working channel 55 of an endoscope 50 (shown schematically in FIG. 1) such that the stent 70 disposed at the distal end region 28 of the catheter 12 reaches a treatment site. Once advanced to the treatment site, the stent delivery system 10 is positioned in such a manner that a proximal end region 14 of the catheter 12 extends proximally from the working channel 55 of the endoscope 50. In some instances a portion of the proximal end region 14 of the catheter 12, including a proximalmost end portion of the inner shaft 20 and/or a proximalmost end portion of the outer tubular shaft 30 may be intraoperatively removed (e.g., cut off, decoupled, etc.) leaving a desired length of the proximal end region 14 of the catheter 12 extending from the endoscope (e.g., extending out of an access port of the endoscope 50). An access port of the endoscope 50 providing access to the working channel 55 may serve as the fixed anchor point for the stent delivery system 10 by preventing any inadvertent movement (e.g., distal and/or proximal movement) of the inner shaft 20 once the distal end 106 of the handle casing 104 is immobilized (fixed to, secured to, in abutment with, etc.) relative to the working channel 55 of the endoscope 50.

The handle 100 may also include a stop 110, which may be positioned at the proximal end 102 of the handle 100. However, it is noted that in other instances the stop 110 may be located distal of the proximal end 102 of the handle 100, such as within a medial region of the handle 100, if desired. The stop 110 may be configured to immobilize the inner shaft 20 to prevent proximal movement of the inner shaft 20 relative to the stop 110, and thus prevent proximal movement of the inner shaft 20 relative to the handle casing 104. For instance, the stop 110 may include an inner ring 112 configured to abut a proximal end surface of the inner shaft 20. In some embodiments, the inner ring 112 of the stop 110 may also include a central opening configured to receive the guidewire 108 therethrough such that the guidewire 108 may extend through the handle 100 and the inner shaft 20, with a proximal end region of the guidewire 108 extending proximal of and exterior of the handle 100.

To ensure accurate deployment of the stent 70 and minimize trauma to the patient, it is important that the sole movement during deployment be the proximal retraction of the outer tubular shaft 30, thus creating a need to keep the other components of the stent delivery system 10, like the inner shaft 20, fixed in position.

Once the catheter 12 is advanced through the working channel 55 to position the stent 70 at a desired treatment site, the proximal end region 14 of the catheter 12 may then be positioned within the lumen 115 of a handle casing 104. In other words, the handle 100 may be provided separately and/or uncoupled to the catheter 12, and then coupled to the catheter 12 intraoperatively. The catheter 12 may be positioned in such a way that the proximal end of the inner shaft 20 abuts the stop 110 disposed within the lumen 115 of the handle casing 104. The proximal end of the outer tubular shaft 30 may be initially positioned in the lumen 115 of the handle casing 104 distal of the stop 110. In this position, the outer tubular shaft 30 may be retracted proximally relative to the inner shaft 20 as the outer tubular shaft 30 passes over the inner shaft 20, through an annular gap 202 of the stop 110, such that a proximal end region of the outer tubular shaft 30 may be advanced proximal of the stop 110.

In some embodiments, the catheter 12 may be cut to adjust its length after positioning the stent 70 at the target location. For example, a proximalmost end portion of the inner shaft 20 and/or a proximalmost end portion of the outer tubular shaft 30 may be intraoperatively removed (e.g., cut off, decoupled, etc.) leaving a desired length of the proximal end region 14 of the catheter 12 extending from the endoscope (e.g., extending out of an access port of the endoscope 50). When the length of the catheter 12 extending proximal of the access port of the endoscope 50 is appropriately established (e.g., but cutting off a proximalmost end portion of the catheter 12), the proximal end of the inner shaft 20 may be aligned with and/or in abutment with a distal surface 114 of the stop 110 (e.g., a distal surface of the inner ring 112 while the distal end of the handle 100 (e.g., a distal end of the handle casing 104) is in contact with (e.g., in abutment with, secured to, etc.) the endoscope 50 (e.g., the access port of the endoscope 50). This allows for intraoperative adjustment of the handle length.

The handle 100 may include a retraction member 40 (e.g., actuator) couplable to the outer tubular shaft 30 to retract the outer tubular shaft 30 proximally relative to the handle casing 104 and the inner shaft 20. The retraction member 40 may be secured to the proximal end region 34 of outer tubular shaft 30 to better enable proximal retraction. In the illustrated example, the retraction member 40 is a sliding knob coupled to the outer tubular shaft 30 by a clamp 42. Other types of retraction members 40, such as a spring-loaded push button, a lever, a threaded coupler, a collar, a collet, or the like, may be utilized to retract the outer tubular shaft 30. Other methods and coupling structures may also be utilized to secure the chosen retraction member 40 onto the outer tubular shaft 30 in accordance with the preferences of the user and/or manufacturer. The retraction member 40, may be longitudinally slidable along a channel 45 of the handle casing 104. For example, the retraction member 40 may be positioned at any location along the proximal end region 34 of the outer tubular shaft 30. In the illustrated example, the retraction member 40 is positioned at a location just proximal of the working channel 55 of the endoscope 50 at is distalmost position to maximize the amount of proximal travel needed to release the stent 70 from its compressed, delivery position, reducing the need to increase the length of the handle 100.

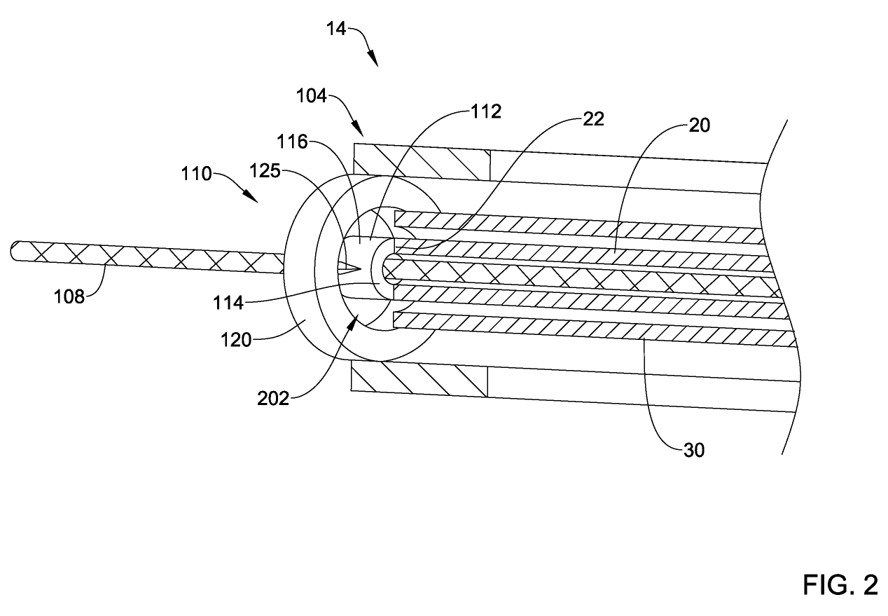

FIG. 2 shows an enlarged view of the proximal end region 14 of the catheter 12 with the positioned handle 100. The inner shaft 20 and the outer tubular shaft 30 are shown in cross-section to better illustrate other aspects of the handle 100. As shown in FIG. 2, the stop 110 may include a distal facing surface 114 configured to contact, abut, or otherwise fix the inner shaft 20 in its position once the proximal end 22 of the inner shaft 20 abuts against the stop 110. Thus, the stop 110 may prevent proximal actuation or movement of the inner shaft 20 proximal of the stop 110. In this position, the outer tubular shaft 30 may still proximally retract relative to the inner shaft 20 and is configured to proximally retract through and/or past the stop 110 while the inner shaft 20 remains fixed in position against the distal facing distal surface 114 of the stop 110.

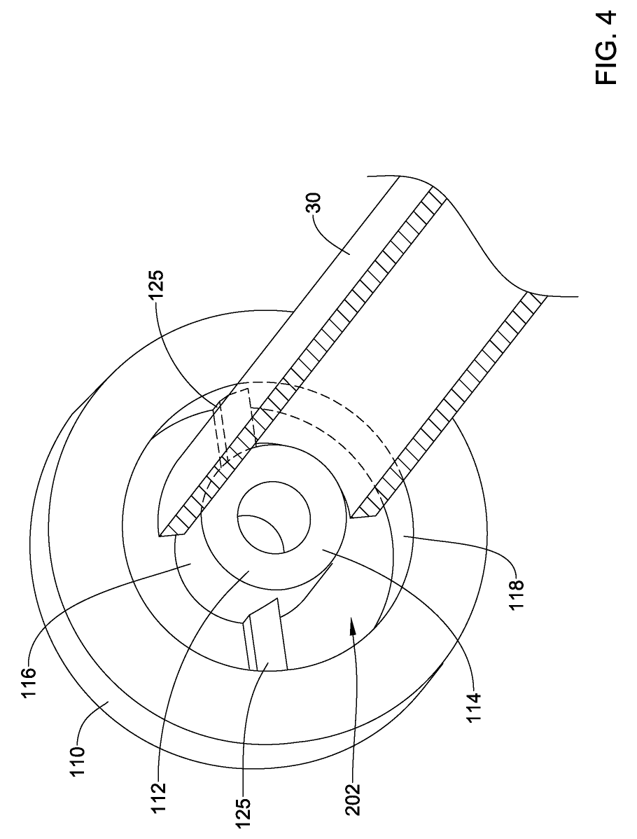

In the illustrated embodiment, the stop 110 is supported in its position within the lumen 115 of the handle casing 104 by at least one strut 125 such that the outer surface 116 of the stop 110 (e.g., a circumferential surface of the stop 110) is spaced radially inward from the interior surface of the handle casing 104, creating the annular gap 202 therebetween. In some embodiments, as illustrated in FIG. 4, the stop 110 may include a plurality of struts 125 suspending the stop 110 within the interior of the handle casing 104. For example, as shown in FIG. 4, the stop 110 may include two struts 125, in which the two struts 125 are diametrically opposed and evenly or symmetrically spaced circumferentially around the stop 110 within the interior surface of the handle casing 104. In other embodiments with two or more struts 125, the struts 125 may be unevenly or asymmetrically spaced. In some embodiments, the strut 125 may include a blade feature having a distally facing sharpened and/or pointed tip configured to cut the outer tubular shaft 30 during retraction of the outer tubular shaft 30 past the strut 125.

In some embodiments, the stop 110 may include a support ring 120 positioned against the interior surface of the handle casing 104. In this embodiment, the strut(s) 125 connect the inner ring 112 of the stop 110 and the support ring 120 to support the position of the inner ring 112 to further a secure the fixed position of the inner shaft 20. In other words, the strut(s) 125 may suspend the inner ring 112 coaxially with the support ring 120, with the annular gap 202 therebetween. Thus, the inner ring 112 may be positioned concentric with the support ring 120, with a circumferential outer surface 116 of the inner ring 112 spaced apart from the circumferential inner surface 118 of the support ring 120, defining the annular gap 202 therebetween.

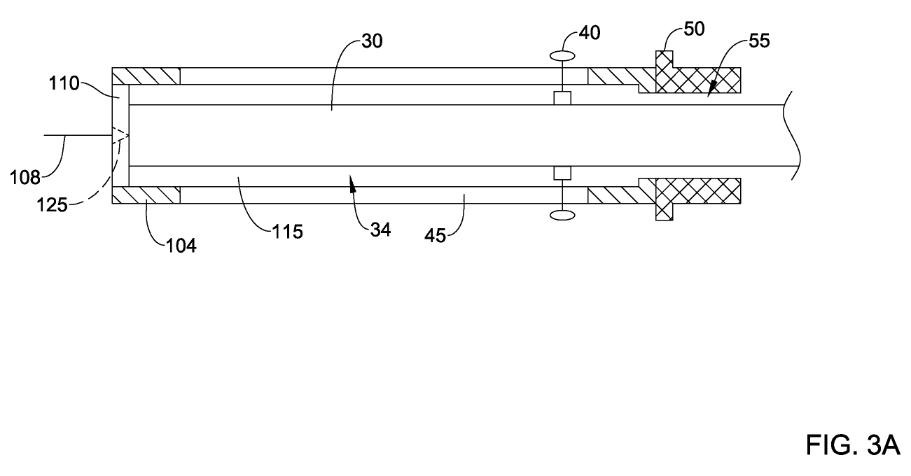

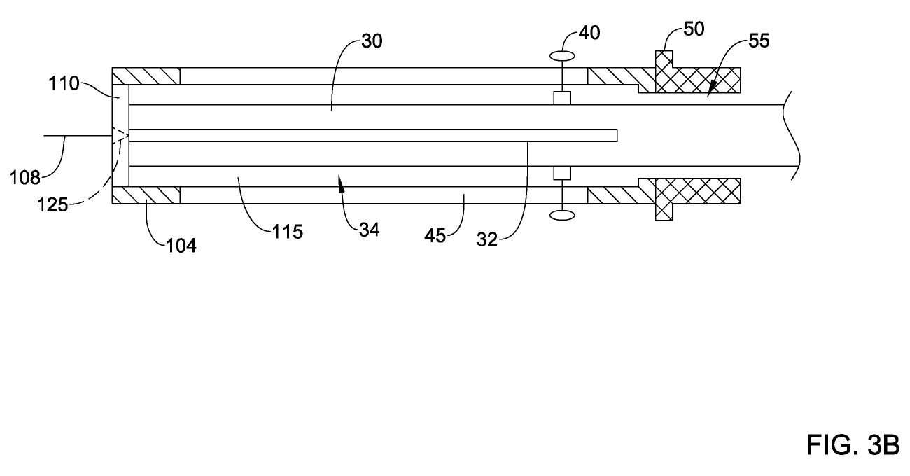

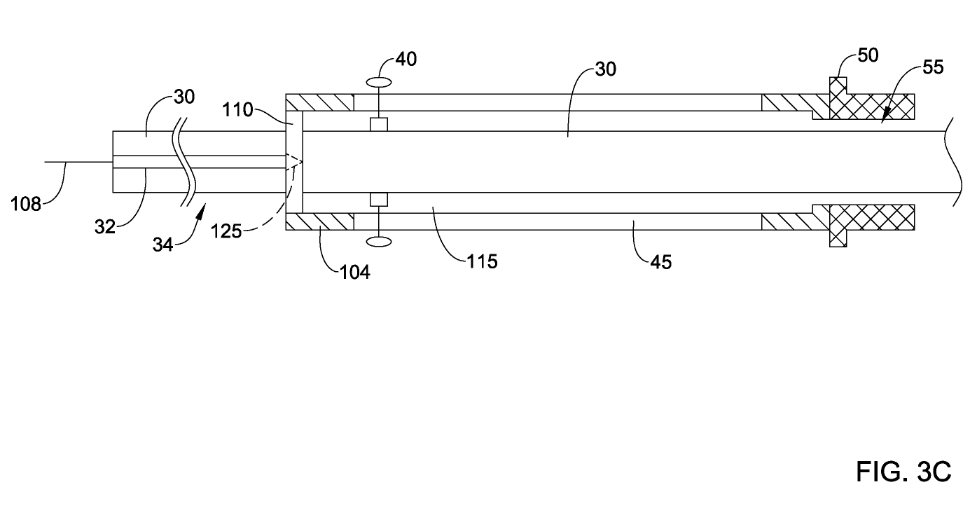

FIG. 3A is a side view, with the handle casing 104 shown in cross-section, illustrating the retraction member 40 in its distalmost position, or pre-deployment position, with the system ready for deployment of the stent 70. FIG. 3B is an alternate side view, with the handle casing 104 shown in cross-section, also illustrating the retraction member 40 in its distalmost position, or pre-deployment position. In the embodiment of FIG. 3B, the outer tubular shaft 30 may be provided with a longitudinal slit or slot 32 extending axially along the proximal end region 34 of the outer tubular shaft 30. The longitudinal slit or slot 32 may be circumferentially aligned with the strut 125 to accommodate the strut 125 once the outer tubular shaft 30 is retracted proximally. The longitudinal slit or slot 32 allows the outer tubular shaft 30 to proximally retract past the stop 110 when deploying the stent 70. In other words, the outer tubular shaft 30 may include at least one slit or slot 32 along its longitudinal axis to allow the outer tubular shaft 30 to pass proximally over the inner ring 112 of the stop 110, through the annular gap 202 as the outer tubular shaft 30 is being proximally retracted relative to the inner shaft 20 (not illustrated, see FIG. 3B). The length of the slit or slot 32 may be sufficient to accommodate the amount of proximal retraction needed for the outer tubular shaft 30 to expose the stent 70 (not illustrated, FIG. 3B) from its constrained, delivery position at the end of the inner shaft 20. The slit or slot 32 may be formed during proximal retraction of the outer tubular shaft 30 or pre-formed. In embodiments in which the stop 110 includes a plurality of struts 125, the outer tubular shaft 30 may include a slit or slot 32 for each strut 125 and circumferentially aligned therewith.

After advancement of the stent 70 to a target site and positioning of the handle 100 on the proximal end region of the catheter 12, the stent delivery system 10 is prepared for deployment of the stent 70. The outer tubular shaft 30 is configured to retract proximally relative to the inner shaft 20 to uncover the stent 70 and permit the stent 70 to radially expand to an expanded state at the target site. When proximally retracted, the outer tubular shaft 30 must travel a sufficient axial distance to expose the previously-constrained stent 70 and release the expanded stent 70 at the treatment site.

To deploy the stent 70 in this manner, the user may utilize the retraction member 40 or other means of actuating the outer tubular shaft 30 proximally relative to the inner shaft 20. As shown in FIG. 3C, the outer tubular shaft 30 may be retracted proximally such that the proximal end of the outer tubular shaft 30 moves proximal of the stop 110, and may extend proximal of the handle 100. Proximal retraction of the outer tubular shaft 30 may be performed while the handle casing 104 remains in engagement with (e.g., in abutment with, secured to, or otherwise in contact with) the access port of the endoscope 50 (shown in FIG. 1).

As the outer tubular shaft 30 is proximally retracted back through the annular gap 202 of the stop 110, the proximal end of the outer tubular shaft 30 is moved proxi8mal of the strut(s) 125 and inner ring 112 of the stop 110. In embodiments in which the strut 125 includes a sharpened blade, the bladed strut 125 cuts through the wall thickness of the outer tubular shaft 30, as the outer tubular shaft 30 passes the strut 125. Each bladed strut 125, whether there is a single strut 125 present, or a plurality of struts 125, creates a corresponding slit 32 in wall of the outer tubular shaft 30 along the length of the outer tubular shaft 30 extending proximal of the strut 125. In this embodiment, the material of the outer tubular shaft 30 may be selected to balance cutability and structural stability of the outer tubular shaft 30.

Alternatively in embodiments in which the outer tubular shaft 30 may include one or more pre-formed slits or slot(s) 32, the strut 125 may extend through the slit or slot 32 as the outer tubular shaft 30 is moved proximal of the strut 125. In this embodiment, the quantity and position of pre-formed slits or slots 32 may correspond to the quantity and position of the struts 125. Thus, in the proximally retracted, deployed configuration of FIG. 3C, the proximal end of the outer tubular shaft 30 is moved proximally past the strut 125 with a proximal end region of the outer tubular shaft 30 passing through the annular gap 202 and surrounding the inner ring 112. The wall thickness of the outer tubular shaft 30 may be smaller than the radial distance of the annular gap 202 to permit clearance for the outer tubular shaft 30 to pass therethrough. Proximal retraction of the outer tubular shaft 30 may be accomplished while the inner shaft 20 remains stationary with the proximal end 22 of the inner shaft 20 abutting the distal face of the inner ring 112.

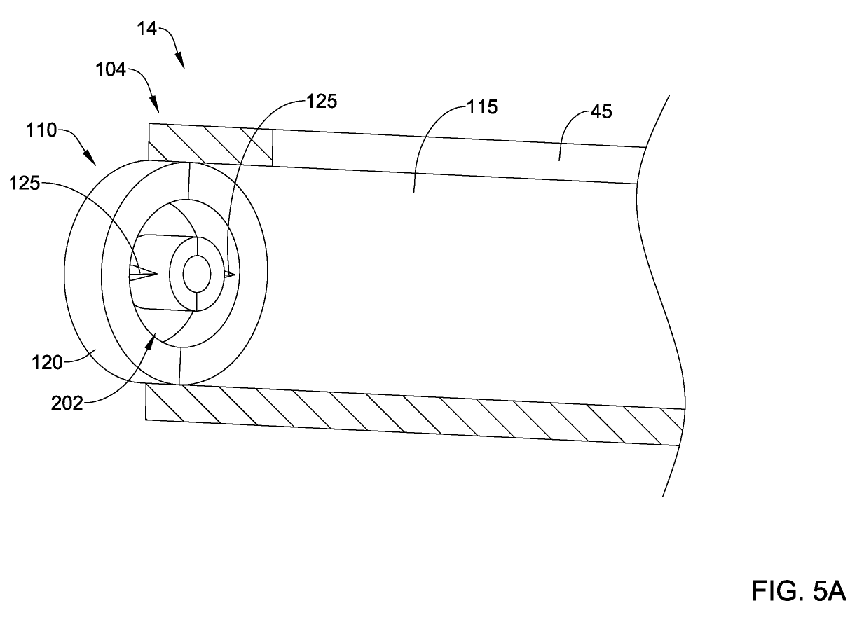

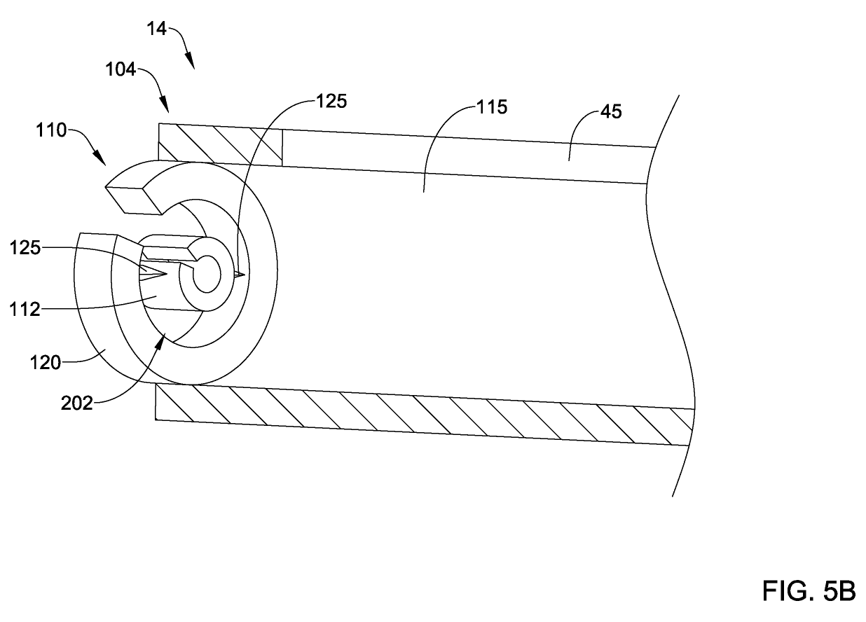

FIG. 5A shows a partial perspective view of one embodiment of the handle 100, in which the handle casing 104 is formed as a clamshell structure. The handle casing 104 can be configured as a clamshell structure for lateral placement of the stent delivery catheter 12 into the lumen 115 of the handle casing 104. In this embodiment, the handle casing 104 may feature a hinged mechanism, snap-fit mechanism, or other equivalent mechanism, allowing a first component (e.g., a first half) and a second component (e.g., a second half) of the handle casing 104 to move apart or open to permit access to the lumen 115 of the handle casing 104. For example, the first and second components or halves of the handle casing 104 may pivot open. This configuration allows for access to the lumen 115 of the handle casing 104 from the side rather than the end of the handle casing 104. The clamshell structure of the handle casing 104 may simplify the loading process by eliminating the need to thread the catheter 12 from one end which may require a longer catheter 12. In some instances, the support ring 120 and/or the inner ring 112 may also be formed in two or more components that may be moved apart or separated to open or permit lateral access therethrough, such as provide lateral access for the guidewire 108 to pass laterally therethrough for placement in the guidewire lumen of the inner ring 112.

The handle 100 may be configured in various manners as to enable lateral placement of the stent delivery catheter 12 therein. Another embodiment of the handle 100 is shown in FIG. 5B. As shown in FIG. 5B, the stop 110, such as the support ring 120 and/or the inner ring 112 of the handle 100 may have a c-shaped cross-section, with a gap formed therein. In this example, the guidewire 108 may be laterally placed within the guidewire lumen of the inner ring 112 while still maintaining the inner shaft 20 position abutting the distal surface 114 of the stop 110. The c-shaped cross-section of the stop 110 and the clamshell structure of the handle casing 104 may be used in conjunction to enable lateral placement of the catheter 12 and the guidewire 108.I In other embodiments, any other combination of features, or only one feature may be included.

It should be understood that the clamshell structure and the c-shaped cross-section of the stop 110 are specific embodiments that would allow lateral placement of the catheter 12 and/or the guidewire 108. Alternative shapes may allow for lateral placement and maintain the ability of the inner shaft 20 to abut against the distal surface 114 of the stop 110, enable securing the inner shaft 20 position, and enable the sliding retraction movement of the outer tubular shaft 30.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent delivery system comprising:
a handle having a proximal end and a distal end, the handle comprising:
a handle casing having an interior surface defining a lumen along a longitudinal axis of the handle casing;
a stop disposed within the lumen of the handle casing; and
at least one strut supporting the stop within the lumen of the handle casing such that an outer circumferential surface of the stop is spaced radially inward of and spaced away from the interior surface of the handle casing; and
a stent delivery catheter comprising:
an inner shaft having a proximal end region configured to be positioned within the lumen of the handle casing with a proximal end of the inner shaft abutting a distal facing surface of the stop; and
an outer tubular shaft slidably disposed over the inner shaft, the outer tubular shaft including a proximal end region configured to be positioned within the lumen of the handle casing;
wherein proximal retraction of the outer tubular shaft causes a proximal end of the outer tubular shaft to be retracted proximal of the stop.

2. The system of claim 1, wherein the strut comprises a blade configured to cut the outer tubular shaft when the outer tubular shaft is retracted proximally relative to the inner shaft.

3. The system of claim 1, wherein the outer tubular shaft includes at least one longitudinal slit corresponding to a position of the strut.

4. The system of claim 1, further comprising a retraction member coupled to the outer tubular shaft.

5. The system of claim 4, wherein the retraction member comprises a sliding knob coupled to the outer tubular shaft by a clamp.

6. The system of claim 1, wherein the stop comprises an inner ring including a central opening configured to receive a guidewire therethrough.

7. The system of claim 1, wherein the handle casing comprises a distal end configured to be secured to a working channel of an endoscope.

8. The system of claim 1, wherein the handle casing is configured for lateral placement of the stent delivery catheter therein.

9. The system of claim 8, wherein the stop has a C-shaped cross-section and a lateral opening.

10. The system of claim 1, wherein the catheter shaft is configured to be cut to adjust its length.

11. The system of claim 1, wherein the at least one strut comprises two diametrically opposed struts.

12. The system of claim 1, wherein the stop includes a support ring secured to the handle casing.

13. The system of claim 12, wherein the at least one strut supports an inner ring of the stop coaxially within the support ring.

14. The system of claim 13, wherein an outer circumferential surface of the inner ring is spaced apart from an inner circumferential surface of the support ring, defining an annular gap therebetween.

15. A stent delivery system comprising:
a handle having a proximal end and a distal end, the handle comprising:
a handle casing having an interior surface defining a lumen along a longitudinal axis of the handle casing;
an inner ring disposed within the lumen of the handle casing, the inner ring including a central opening configured to receive a guidewire therethrough; and
at least one strut supporting the inner ring within the lumen of the handle casing such that an outer diameter of the inner ring is spaced radially inward of the inner surface of the handle casing;
a stent delivery catheter comprising:
an inner shaft; and
an outer tubular shaft slidably disposed over the inner shaft;
wherein a proximal end of the inner shaft is configured to abut the inner ring within the lumen of the handle casing; and
wherein the outer tubular shaft is configured surround the inner ring when the outer tubular shaft is retracted proximally relative to the inner shaft.

16. The system of claim 15, wherein the strut comprises a blade configured to cut the outer tubular shaft when the outer tubular shaft is retracted proximally relative to the inner shaft.

17. The system of claim 15, wherein the outer tubular shaft includes at least one pre-formed longitudinal slit corresponding to a position of the strut, configured to allow the outer tubular shaft to pass over the inner ring during proximal retraction of the outer tubular shaft relative to the inner shaft.

18. The system of claim 15, further comprising a support ring secured to the handle casing, wherein the at least one strut supports the inner ring coaxially within the support ring.

19. The system of claim 15, wherein a proximal end of the inner shaft is configured to abut the inner ring within the lumen of the handle casing.

20. A method of deploying a stent comprising:
advancing a catheter shaft to a treatment site through a working channel of an endoscope, wherein a proximal end region of the catheter shaft extends proximally from the working channel of the endoscope, the catheter shaft comprising:
an inner shaft having a distal end, a proximal end, and a lumen configured to receive a guidewire; and
an outer tubular shaft, having a distal end and a proximal end, slidably disposed over the inner shaft, wherein the outer tubular shaft is configured to hold a stent in a constrained position on the distal end of the inner shaft;
positioning the proximal end region of the catheter shaft into a lumen of a handle casing of a handle such that the proximal end of the inner shaft abuts a distally facing stop within the lumen of the handle casing;
securing a distal end of the handle casing to the working channel of the endoscope; and
retracting the outer tubular shaft proximally relative to the inner shaft to deploy the stent.
